(19) United States
(12) Patent Application Publication
(10) Pub. No.: US 2003/0088170 A1
(43) Pub. Date: May 8, 2003

(54) Non-contact tonometer having mechanically isolated cylinder

(75) Inventors: Bruce Siskowski, Orchard Park, NY (US); David A. Luce, Clarence Center, NY (US)

Correspondence Address: Simpson, Simpson & Snyder, PLLC, 5555 Main Street, Williamsville, NY, 14221, US

(73) Assignee: Leica Microsystems Inc., Depew, NY

(21) Appl. No.: 09/993,281

(22) Filed: Nov. 6, 2001

(51) Int. Cl. A61B 3/16

(52) U.S. Cl. 600/399; 600/401

(57) ABSTRACT

A non-contact tonometer comprises a fluid pump system configured and mounted to dissipate vibration energy to reduce the effect of vibrations on measurement components caused by the stroke of a piston with respect to a cylinder in the fuid pump system. In a preferred embodiment, a compression chamber receiving a piston and plenum chamber containing a pressure sensing device are spaced apart from one another and connected by a flow tube formed of a vibration damping material, and at least one vibration damping element is provided between the cylinder and a support frame of the non-contact tonometer.

### BACKGROUND OF THE INVENTION

I. Field of the Invention

The present invention relates generally to ophthalmic instruments, and more particularly to a non-contact tonometer having an improved fluid pump apparatus that reduces measurement-affecting vibrations.

II. Description of the Related Art

Non-contact tonometers are well-known in the field of ophthalmology for measuring intraocular pressure (IOP) by directing a fluid pulse at the cornea to cause observable deformation of the cornea. In prior art non-contact tonometers, such as tonometer 10 shown schematically in FIG. 1, the fluid pulse is generated by a piston 12 slidably received by a cylinder housing 14 and axially driven relative to the cylinder housing to compress fluid within a compression chamber 16 defined by the cylinder housing. A plenum chamber 18 directly adjoins compression chamber 14, and a fluid discharge tube 20 is arranged in flow communication with the compression chamber by way of the plenum chamber for directing a fluid pulse along a test axis TA toward cornea C. The piston 12 is typically driven by automatic drive means 22, for example a linear motor or a rotary solenoid connected to the piston by a linkage, wherein the drive means is energized by a current source 24 under the control of a microprocessor 26. Signal information from a pressure sensor 28 located in the plenum chamber 18, and signal information from a photosensitive applanation detector 30 cooperating with an emitter 32 mounted in a nosepiece 34, are digitized by analog-to-digital converter circuits 29 and input to the microprocessor 24 for calculating IOP. Cylinder housing 14 and nosepiece 34 are fixedly mounted on a support frame 11 of tonometer 10.

During its measurement stroke, piston 12 is accelerated very rapidly from rest to generate a fluid pulse of very short duration, and then is decelerated very rapidly and forced to move in a reciprocal direction to its start or reference position. As can be understood, the piston stroke is accompanied by vibrations that are propagated through the cylinder housing to other parts of the instrument, including pressure sensor 28, nosepiece 34, and applanation detector 30 and emitter 32 carried by the nosepiece. Consequently, these vibrations have an undesirable effect on the measurement accuracy of the instrument.

### BRIEF SUMMARY OF THE INVENTION

Therefore, it is an object of the present invention to design a non-contact tonometer wherein vibration propagation associated with the piston stroke is reduced.

In accordance with the present invention, this object is achieved by physically separating the compression chamber from the plenum chamber containing the pressure sensing device, and connecting the two chambers via a flow tube preferably formed of a vibration damping material. As a further aspect of the present invention, at least one vibration damping element is provided between the cylinder and a support frame of the non-contact tonometer to limit vibration transfer to the support frame and other instrument components mounted on the support frame.

### BRIEF DESCRIPTION OF THE SEVERAL VIEWS OF THE DRAWINGS

The nature and mode of operation of the present invention will now be more fully described in the following detailed description of the invention taken with the accompanying drawing figures, in which:

FIG. 1 is a schematic depiction of a fluid pump system for a non-contact tonometer formed in accordance with known prior art; and

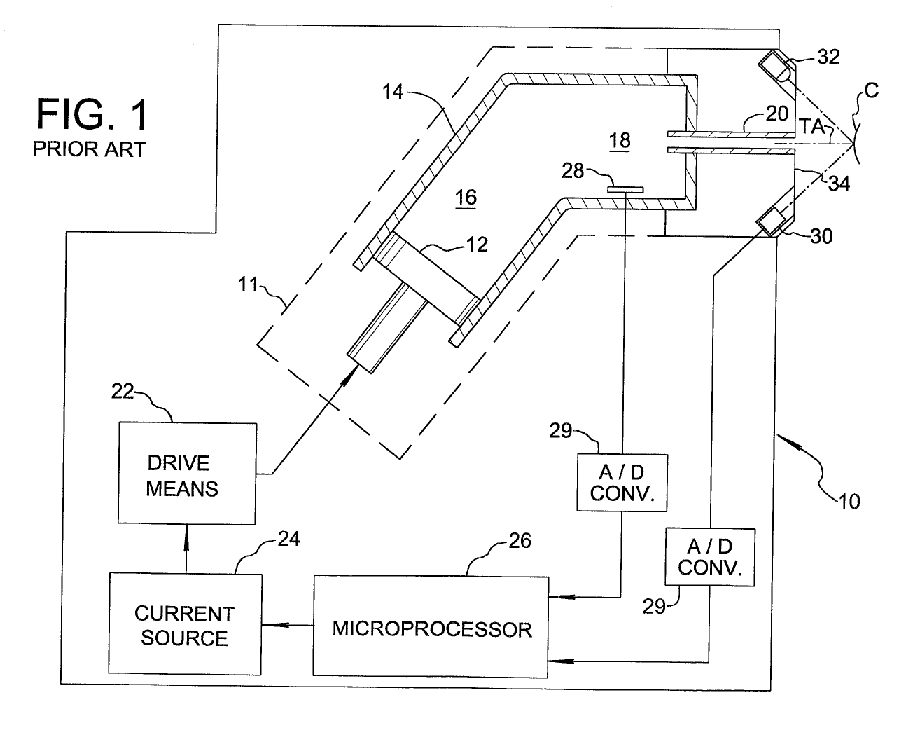

FIG. 2 is a schematic depiction of a fluid pump system for a non-contact tonometer formed in accordance with a preferred embodiment of the present invention.

### DESCRIPTION OF THE INVENTION

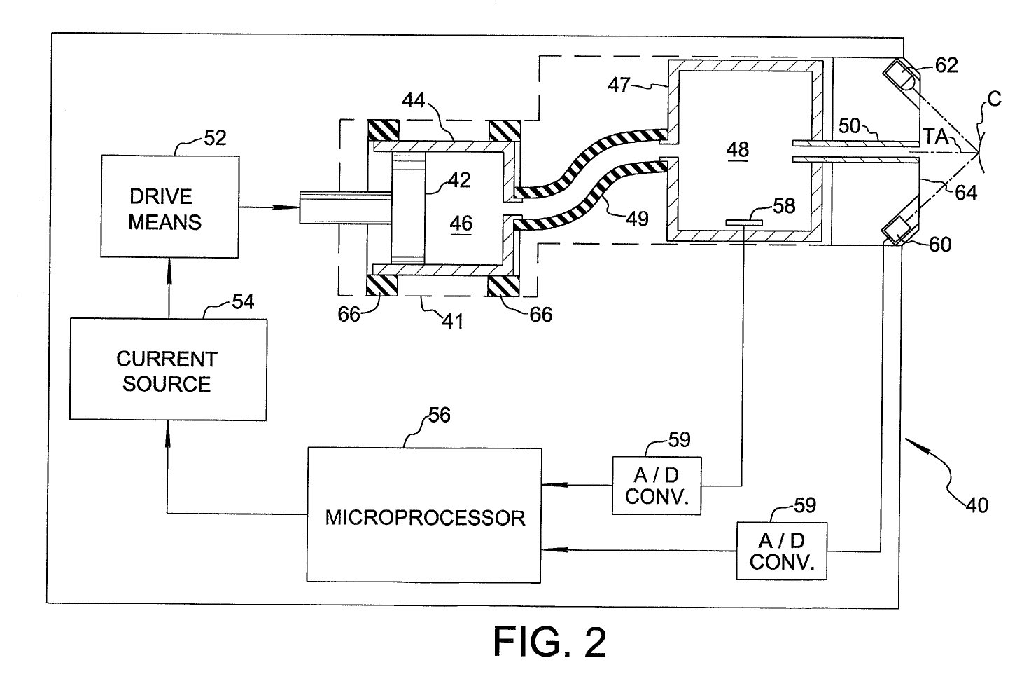

Referring to FIG. 2 of the drawings, a tonometer 40 includes a fluid pump system for generating a fluid pulse used to flatten or “applanate” a patient's cornea during testing. In accordance with a preferred embodiment of the present invention, the fluid pump system comprises a piston 42 axially movable relative to a cylinder 44 for compressing fluid within an internal compression chamber 46 defined thereby, an isolation housing 47 defining an internal plenum chamber 48, a flow tube 49 providing a fluid conduit from compression chamber 46 to plenum chamber 48, and a fluid discharge tube 50 mounted through the wall of isolation housing 47 for guiding pressurized fluid from plenum chamber 48 along test axis TA directed at patient cornea C. An electromotive drive 52, such as a solenoid or electric motor, is operatively connected to piston 42 for causing axially directed movement of piston 42 relative to cylinder 44. electromotive drive 52 is energized by current supplied by a current source 54 under the control of a microprocessor 56.

A pressure sensing device 58, for example a pressure transducer or the like, is located within plenum chamber 48 for generating signal information indicative of the fluid pressure within the plenum chamber. Pressure sensing device 58 is connected to microprocessor 56 by way of an analog-to-digital converter 59, and the microprocessor receives and processes the digitized pressure signal information for measurement purposes.

A photosensitive detector 60 and an emitter 62 are positioned on opposite sides of test axis TA such that light from emitter 62 is reflected by the flattened surface of applanated cornea C in the direction of detector 60, causing the detector to generate a peak signal at the moment of applanation. Signal information from applanation detector 60 is delivered to microprocessor 56 via an analog-to-digital converter 59, and the microprocessor receives and processes the digitized applanation signal information along with the pressure signal information to provide a measurement value of IOP.

The applanation detection optics, namely emitter 62 and detector 60, are fixedly mounted in a nosepiece 64. Cylinder 44, isolation housing 47, and nosepiece 64 are carried by a support frame 41 of tonometer 40.

It will be appreciated from the above description that isolation housing 47 is physically remote from cylinder 44, and thus is not exposed to local cylinder vibrations associated with the movement of piston 42. Since pressure sensing device 58 is located in plenum chamber 48 of isolation housing 47, it is substantially protected from vibrations local to cylinder 44 that could affect the pressure signal and compromise measurement accuracy.

Another aspect of the present invention is the use of a vibration damping material in the construction of flow tube 49 to prevent transmission of vibrations from cylinder 44 to isolation housing 47. Preferably, at least a portion of flow tube 49 is formed of a vibration damping material, such as synthetic rubber, to dissipate vibration energy before it reaches isolation housing 47. In a presently preferred construction, the entire flow tube 49 is formed of polyurethane.

A further aspect of the present invention is the use of at least one vibration damping element 64 operatively arranged between cylinder 44 and support frame 41 for dissipating vibration energy. In an embodiment preferred for its simplicity, a pair of vibration damping elements 66 are configured as rings formed of a vibration damping material fitted circumferentially about cylinder 44 at opposite axial ends thereof. Suitable vibration damping material for forming damping elements 66 is synthetic rubber, for example polyurethane, however other vibration damping materials can be used. It is noted that vibration damping elements 66 can be provided for mounting cylinder 14 on surrounding support frame 41 even when no physically remote isolation housing 47 is provided, whereby some benefit is nevertheless realized.

What is claimed is:

1. In a non-contact tonometer having a cylinder defining a compression chamber, a piston movable relative to said cylinder for compressing fluid within said compression chamber, and a fluid discharge tube in flow communication with said compression chamber for directing a fluid pulse along an axis, the improvement comprising:
an isolation housing spaced from said cylinder, said isolation housing defining an internal plenum chamber; and
a flow tube providing flow communication between said compression chamber and said plenum chamber.

2. The improvement according to claim 1, wherein said flow tube is formed of a vibration damping material.

3. The improvement according to claim 2, wherein said vibration damping material is a synthetic rubber.

4. The improvement according to claim 3, wherein said vibration damping material is polyurethane.

5. The improvement according to claim 1, wherein said fluid discharge tube is supported by said isolation housing and is arranged for flow communication with said plenum chamber.

6. The improvement according to claim 1, further comprising a pressure sensing device located in said plenum chamber.

7. The improvement according to claim 6, wherein said pressure sensing device is a pressure transducer.

8. In a non-contact tonometer having a support frame, a cylinder connected to said support frame and defining a compression chamber, and a piston movable relative to said cylinder for compressing fluid within said compression chamber, the improvement comprising:
at least one vibration damping element operatively arranged between said cylinder and said support frame.

9. The improvement according to claim 8, wherein said at least one vibration damping element comprises a ring of vibration damping material arranged circumferentially about said cylinder.

10. The improvement according to claim 9, wherein said at least one vibration damping element comprises a pair of rings of vibration damping material arranged circumferentially about said cylinder at opposite axial ends thereof.

11. The improvement according to claim 10, wherein said vibration damping material is a synthetic rubber.

12. The improvement according to claim 11, wherein said vibration damping material is polyurethane.

13. A fluid pump system for a non-contact tonometer, said fluid pump system comprising:
a cylinder defining a compression chamber;
a piston movable relative to said cylinder for compressing fluid within said compression chamber;
an isolation housing spaced from said cylinder, said isolation housing defining an internal plenum chamber;
a flow tube providing flow communication between said compression chamber and said plenum chamber; and
a fluid discharge tube communicating with said plenum chamber for directing a fluid pulse along an axis.

14. The fluid pump system according to claim 13, wherein said flow tube is formed of a vibration damping material.

15. The fluid pump system according to claim 14, wherein said vibration damping material is a synthetic rubber.

16. The fluid pump system according to claim 15, wherein said vibration damping material is polyurethane.

17. The fluid pump system according to claim 13, further comprising at least one vibration damping element operatively arranged about said cylinder.

18. The fluid pump system according to claim 17, wherein said at least one vibration damping element comprises a pair of rings of vibration damping material arranged circumferentially about said cylinder at opposite axial ends thereof.

19. The fluid pump system according to claim 18, wherein said vibration damping material is a synthetic rubber.

20. The fluid pump system according to claim 19, wherein said vibration damping material is polyurethane.

21. The fluid pump system according to claim 13, further comprising a pressure sensing device located in said plenum chamber.

22. The improvement according to claim 21, wherein said pressure sensing device is a pressure transducer.
